(19) United States
(12) Patent Application Publication
(10) Pub. No.: US 2004/0023926 A1
(43) Pub. Date: Feb. 5, 2004

(54) Novel organosilicon compounds comprising a multifunctional polyorganosiloxane bearing at least one activated imide-type double ethylene bond and method for preparing same

(76) Inventors: Nathalie Guennouni, Irigny (FR); Jean-Jacques Lebrun, Pierre Benite (FR)

Correspondence Address: BURNS DOANE SWECKER & MATHIS L L P, POST OFFICE BOX 1404, ALEXANDRIA, VA, 22313-1404, US

(21) Appl. No.: 10/311,545

(22) PCT Filed: Jun. 14, 2001
(86) PCT No.: PCT/FR01/01858

(30) Foreign Application Priority Data
Jun. 16, 2000 (FR) 00/07699

(51) Int. Cl. A61K 31/695; A61K 7/06; A61K 7/11

(52) U.S. Cl. 514/63; 548/408; 424/70.122

(57) ABSTRACT

The field of the present invention is that of novel organosilicon compounds comprising a multifunctional polyorganosiloxane (abbreviated as POS) comprising, per molecule, and attached to silicon atoms, firstly at least one hydroxyl radical and/or at least one alkoxy radical, and secondly at least one group containing an activated ethylenic double bond consisting of a maleimide group. The present invention also relates to functionalization processes leading to the POSs targeted above, which consist in particular in reacting an organosilane bearing a maleamic acid function and at least two alkoxy functions with a polysilazane.

The compounds comprising a multifunctional POS as targeted above are capable of showing advantageous properties, for example of acting as coupling agents (for white filler-elastomer coupling) in rubber compositions based on isoprene elastomer(s) comprising a white filler as reinforcing filler.

The field of the present invention is that of novel organosilicon compounds comprising a multifunctional polyorganosiloxane (abbreviated as POS) comprising, per molecule, and attached to silicon atoms, firstly at least one hydroxyl radical and/or at least one alkoxy radical, and secondly at least one group containing an activated ethylenic double bond consisting of a maleimide group. The present invention also relates to functionalization processes leading to the POSs targeted above.

The compounds comprising a multifunctional POS as targeted above are capable of showing advantageous properties, for example of acting as coupling agents (for white filler-elastomer coupling) in rubber compositions based on isoprene elastomer(s) comprising a white filler as reinforcing filler.

The principle of multifunctionalization of POSs is disclosed, for example, in document WO-A-96/16125 in the name of the Applicant, which discloses the preparation of multifunctional POSs bearing ≡Si—O-alkyl functional units and ≡Si—W functional units in which W is in particular a C₂-C₃₀ hydrocarbon-based group, a simple alkyenyl group, an unsaturated cycloaliphatic group or a mercaptoalkyl group.

In continuation of studies in the field of multifunctionalization, the Applicant has now found, and this constitutes the first subject of the invention, novel multifunctional POSs bearing, in addition to at least one alkoxy and/or hydroxyl radical, at least one maleimide group.

The maleimide group is found to be an advantageous function in chemical processes in which reactions towards active species such as, for example, a hydrocarbon-based radical C•, a mercaptoalkyl radical RS•, a mercaptoalkyl anion RS⁻, and cycloaddition reactions (“ene” reactions) are involved in particular.

The review of the prior art reveals that synthetic methods for gaining access to maleimide functions are varied. However, the Applicant has found that the synthetic methods usually proposed, when applied to silicone chemistry, may not offer satisfactory functionalization yields when, as is frequently the case, the operating conditions used substantially modify the silicone skeleton and consequently proportionally minimize the selectivity of the synthetic method; these drawbacks are encountered for the processes disclosed, in particular, in documents FR-A-2 295 959 and FR-A-2 308 126. Another object of the present invention is thus to provide processes for preparing POSs bearing maleimide function(s) which are easy to carry out and which offer the undeniable advantage of giving functionalized POSs with selectivities, stabilities and yields in an excellent level which has not yet been achieved hitherto.

### FIRST SUBJECT OF THE INVENTION

Consequently, the present invention, taken in its first subject, relates to organosilicon compounds which comprise multifunctional POSs containing identical or different units of formula:

$${( {R}^{2} )}_{a} {Y}_{b} {X}_{c} {SiO}_{\frac{4 - ( a + b + c )}{2}} \qquad ( I )$$

in which:

(1) the symbols R², which may be identical or different, each represent a monovalent hydrocarbon-based group chosen from a linear or branched alkyl radical containing from 1 to 6 carbon atoms, a cycloalkyl radical containing from 5 to 8 carbon atoms and a phenyl radical; preferably, the symbols R² are chosen from methyl, ethyl, n-propyl, isopropyl, n-butyl, n-pentyl, cyclohexyl and phenyl radicals; more preferably, the symbols R² are methyl radicals;

(2) the symbols Y, which may be identical or different, each represent a hydroxyl or alkoxy function R¹O in which R¹ represents a linear or branched alkyl radical containing from 1 to 15 carbon atoms; preferably, the symbols Y are chosen from a hydroxyl radical and a linear or branched alkoxy radical containing from 1 to 6 carbon atoms; more preferably, the symbols Y are chosen from a hydroxyl radical and a linear or branched alkoxy radical containing from 1 to 3 carbon atoms (that is to say methoxy, ethoxy, propoxy and/or isopropoxy);

(3) the symbols X, which may be identical or different, each represent a function bearing an activated ethylenic double bond, chosen from the radicals having the formulae (II/1), (II/2) and (II/3) below, and mixtures thereof:

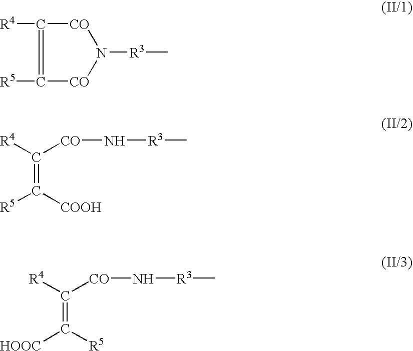

with the conditions according to which:

at least one of the functions X corresponds to formula (II/1),

when, where appropriate, there is a mixture of function(s) X of formula (II/1) with functions X of formulae (II/2) and/or (II/3), the mole fraction of functions X of formulae (II/2) and/or (II/3) in all of the functions X is on average less than or equal to 12 mol % and preferably less than or equal to 5 mol %,

formulae in which:

R³ is a linear or branched divalent alkylene radical containing from 1 to 15 carbon atoms, the free valency of which is borne by a carbon atom and is linked to a silicon atom, the said radical R³ possibly being interrupted in the alkylene chain with at least one hetero atom (such as oxygen and nitrogen) or at least one divalent group comprising at least one hetero atom (such as oxygen and nitrogen), and in particular with at least one divalent residue of general formula ^{V1}residue^{V2}chosen from: —O—, —CO—, —CO—O—, —COO-cyclohexylene (optionally substituted with an OH radical)-, —O-alkylene (linear or branched C₂-C₆, optionally substituted with an OH or COOH radical)-, —O—CO-alkylene (linear or branched C₂-C₆, optionally substituted with an OH or COOH radical)-, —CO—NH—, O—CO—NH— and —NH-alkylene (linear or branched C₂-C₆)—CO—NH—; R³ also represents a divalent aromatic radical of general formula ^{V1}radical^{V2}chosen from: -(ortho, meta or para)phenylene(linear or branched C₂-C₆)alkylene-, -(ortho, meta or para)phenylene-O-(linear or branched C₂-C₆)alkylene-, -(linear or branched C₂-C₆)alkylene-(ortho, meta or para)phenylene(linear or branched C₁-C₆)alkylene-, and -(linear or branched C₂-C₆)alkylene(ortho, meta or para)phenylene-O-(linear or branched C₁-C₆)alkylene-; preferably, the symbol R³ represents an alkylene radical which corresponds to the following formulae: —(CH₂)₂—, —(CH₂)₃—, —(CH₂)₄—, —CH₂—CH(CH₃)—, —(CH₂)₂—CH(CH₃)—CH₂—, —(CH₂)₃—O—(CH₂)₃—, —(CH₂)₃—O—CH₂—CH(CH₃)—CH₂—, —(CH₂)₃—O—CH₂CH(OH)—CH₂—; more preferably, R³ is a —(CH₂)₂— or —(CH₂)₃-radical; with the specific detail that, in the preceding definitions of R³, when the divalent residues and radicals mentioned are not symmetrical, they may be positioned with the valency v1 to the left and the valency v2 to the right, or vice versa with the valency v2 to the left and the valency v1 to the right;

the symbols R⁴ and R⁵, which may be identical or different, each represent a hydrogen atom, a halogen atom, a cyano radical or a linear or branched alkyl radical containing from 1 to 6 carbon atoms; preferably, the symbols R⁴ and R⁵ are chosen from a hydrogen atom, a chlorine atom and methyl, ethyl, n-propyl and n-butyl radicals; more preferably, these symbols are chosen from a hydrogen atom and a methyl radical;

(4) the symbols a, b and c each represent integers or fractions chosen from:

a: 0, 1, 2 or 3;

b: 0, 1, 2 or 3;

c: 0 or 1;

the sum a+b+c being other than zero and ≦3;

(5) the content of units R⁶SiO_{3/2} (units “T”) in which R⁶ is chosen from the radicals corresponding to the definitions of R², Y and X, this content being expressed as the number, per molecule, of these units per 100 silicon atoms, is less than or equal to 30% and preferably less than or equal to 20%;

(6) the content of functions Y, expressed as the number, per molecule, of functions Y per 100 silicon atoms, is at least 0.8% and is preferably in the range from 1% to 100%;

(7) the content of functions X, expressed as the number, per molecule, of functions X per 100 silicon atoms, is at least 0.4% and is preferably in the range from 0.8% to 100%.

Given the values which symbols a, b and c may take and the details given in point (5), it should be understood that each multifunctional POS of formula(I) may have either a linear structure or a cyclic structure, or a mixture of these structures, these structures also possibly having a certain molar amount of branching (units “T”).

The invention concerns organosilicon compounds “which comprise multifunctional POSs”; this expression should be interpreted as meaning that each organosilicon compound forming part of the present invention may be in the form of a multifunctional POS in pure form or in the form of a mixture of such a POS with a variable weight amount (generally much less than 50% in the mixture) of another (or of other) compound(s) which may consist of:

(i) one and/or other of the starting reagents from which the multifunctional POSs are prepared, when the degree of conversion of the said reagents is not complete; and/or

(ii) the product(s) derived from a complete or incomplete modification of the silicone skeleton of the starting reagent(s); and/or

(iii) the product(s) derived from a modification of the silicone skeleton of the desired multifunctional POS, prepared by a condensation reaction, a hydrolysis and condensation reaction and/or a redistribution reaction.

To be more specific, the organosilicon compounds which are included in the scope of the invention are those which comprise multifunctional POSs chosen from the family of POSs in accordance with formula (I), which are essentially linear and have the average formula below:

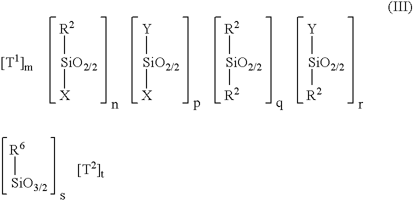

in which:

(1′) the symbols T¹ are chosen from the units HO_{1/2} and R¹O_{1/2}, in which the radical R¹ is as defined above;

(2′) the symbols T², which may be identical to or different from the symbols T¹, are chosen from the units HO_{1/2} and R¹O_{1/2} and the unit (R²)₃SiO_{1/2}, in which the radicals R¹ and R² are as defined above in points (2) and (1) regarding formula (I);

(3′) the symbols R², X and Y are as defined above in points (1), (3) and (2) regarding formula (I);

(4′) the symbols R⁶ are chosen from the radicals corresponding to the definitions of R², X and Y;

(5′) the symbols m, n, p, q, r, s and t each represent integers or fractions which satisfy the following cumulative conditions:

m and t are each numbers that are always other than zero, the sum of which is equal to 2+s,

n is in the range from 0 to 100,

p is in the range from 0 to 100,

q is in the range from 0 to 100,

r is in the range from 0 to 100,

s is in the range from 0 to 75,

when n=0, p is always a number other than 0 and when p=0, n is always a number other than zero,

the sum n+p+q+r+s+t giving the total number of silicon atoms is in the range from 2 to 250,

the ratio 100 s/(n+p+q+r+s+t) giving the content of units “T” is ≦30 and preferably ≦20,

the ratio 100 (m+p+r+s [when R⁶=Y]+t)/(n+p+q+r+s+t) giving the content of functions Y (borne by the units represented by the symbols T¹, T² and Y) is ≧1 and preferably ranges from 4 to 100,

the ratio 100 (n+p+s [when R⁶=X])/(n+p+q+r+s+t) giving the content of functions X is ≧1 and preferably ranges from 2 to 100.

As organosilicon compounds which are preferably used, mention may be made of those comprising the essentially linear oligomers and polymers POS/1 which correspond to formula (III) in which (in this case, these will be referred to for short as polymers POS/1 of imide type):

(1″) the symbols T¹ are defined as given above in point (1′);

(2″) the symbols T² are defined as given above in point (2′);

(3″) the symbols R², X and Y are defined as given above in point (3′);

(4″) the symbols R⁶ are defined as given above in point (4′);

(5″) the symbols m, n, p, q, r, s and t satisfy the following cumulative conditions:

m+t=2+s,

n is in the range from 0 to 50,

p is in the range from 0 to 20,

when n=0, p is at least equal to 1 and when p=0, n is at least equal to 1,

q is in the range from 0 to 48,

r is in the range from 0 to 10,

s is in the range from 0 to 1,

the sum n+p+q+r+s+t giving the total number of silicon atoms is in the range from 2 to 50,

the ratio 100 s/(n+p+q+r+s+t) giving the content of units “T” is ≦10,

the ratio 100 (m+p+r+s [when R⁶=Y]+t)/(n+p+q+r+s+t) giving the content of functions Y (provided by the units represented by symbols T¹, T² and Y) ranges from 4 to 100 and better still from 10 to 100,

the ratio 100 (n+p+s [when R⁶=X])/(n+p+q+r+s+t) giving the content functions X ranges from 10 to 100 and better still from 20 to 100.

Organosilicon compounds which are also included in the scope of the invention are those which comprise multifunctional POSs chosen from the family of POSs in accordance with the formula (I), which are cyclic and have the average formula below:

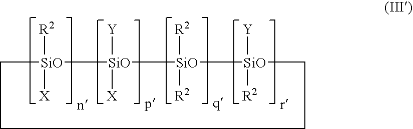

in which:

(3′) the symbols R², X and Y are as defined above in points (1), (3) and (2) regarding formula (I);

(5) the symbols n′, p′, q′ and r′ each represent integers or fractions which satisfy the following cumulative conditions:

n′ is in the range from 0 to 9,

p′ is in the range from 0 to 9,

when n′=0, p′ is at least equal to 1,

when p′=0, n′ is at least equal to 1 and r′ is also at least equal to 1,

q′ is in the range from 0 to 9,

q′ is in the range from 0 to 9,

r′ is in the range from 0 to 2,

the sum n′+p′+q′+r′ is in the range from 3 to 10,

the ratio 100 (p′+r′)/(n′+p′+q′+r′) giving the content of functions Y ranges from 4 to 100,

the ratio 100 (n′+p′)/(n′+p′+q′+r′) giving the content of functions X ranges from 10 to 100.

It should be noted that these cyclic multifunctional POSs may be obtained as a mixture with the essentially linear multifunctional POSs of formula (III).

### SECOND SUBJECT OF THE INVENTION

The second subject of the present invention relates to processes by means of which the organosilicon compounds according to the invention, comprising multifunctional POSs in accordance with formulae (I), (III) and (III″) given above, may be prepared.

These processes in particular involve:

a hydrolysis and condensation reaction of a dihalosilane or of a dialkoxysilane bearing a function X, optionally in the presence of a dihalosilane or of a dialkoxysilane,

a condensation reaction between an organosilane bearing a function X and at least two functions Y, and an α,ω-dihydroxy linear POS,

a redistribution and equilibration reaction between an organosilane bearing a function X and at least two functions Y and/or halo, and an organocyclosiloxane optionally bearing one or more functions Y in the chain,

a coupling reaction between an organosilane bearing a function X of formula (II/2) and at least two functions Y, and a polysilazane,

a coupling reaction between a linear or cyclic precursor POS bearing at least one function Y and functionalized with at least one unit attached to a silicon atom, in particular of -(linear or branched C₂-C₆)alkylene-OH, -(linear or branched C₂-C₆)alkylene-NR⁶H or -(linear or branched C₂-C₆)alkylene-COOH type, and a reactive compound capable of reacting with the abovementioned unit(s) to generate the desired function X.

More specifically, the organosilicon compounds comprising the multifunctional POSs in accordance with formulae (I), (III) and (III′) are prepared by a process which consists, for example:

(a) in hydrolysing, in aqueous medium, an organosilane of formula:

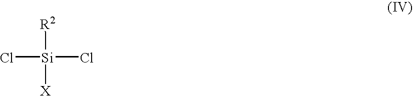

in which the symbols R² and X [which have the formula (II/1)] have the definitions already given above, optionally working in the presence of an organosilane of formula:

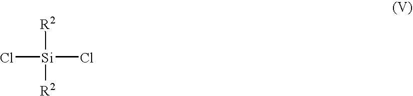

Such a process is suitable for preparing organosilicon compounds comprising multifunctional POSs of formula (III) in which the symbols T¹ and T² each represent the unit HO_{1/2} and in which, firstly, p=r=s=0 and, secondly, q is either equal to zero [when the silane (IV) is hydrolysed in the absence of the silane (V)], or a number other than zero [when the silane (IV) is hydrolysed in the presence of the silane (V)]. As regards the practical method for carrying out this process, reference will be made for further details to the content of FR-A-2 514 013;

(b) in condensing, optionally in the presence of a catalyst based, for example, on a tin carboxylate, an organosilane of formula:

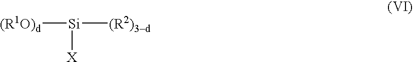

in which the symbols R¹, R² and X [which have the formula (II/1)] are as defined above and d is a number chosen from 2 and 3, with a POS of formula:

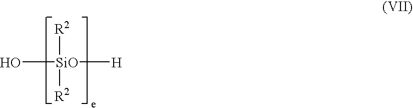

in which the symbol R² is as defined above and e is an integer or fraction ranging from 2 to 50. Such a process is suitable for preparing organosilicon compounds comprising multifunctional POSs of formula (III) in which the symbols T¹ and T² lie in a mixture of units HO_{1/2} with units R¹O_{1/2} and in which the symbols p, r and s may be other than zero when d=3, whereas, irrespective of the value of d, q is other than zero. As regards the practical method for carrying out this process, reference may be made for further details to the content of U.S. Pat. No. 3,755,351;

(c) in carrying out a redistribution and equilibration reaction, in the presence of a suitable catalyst and water, between, on the one hand, an organosilane of formula:

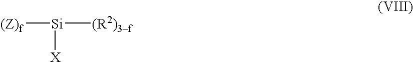

in which the symbols R² and X [which has the formula (II/1)] are as defined above, the symbol Z is chosen from hydroxyl, R¹O and halo (such as, for example, chlorine) radicals and f is a number chosen from 2 and 3, and, on the other hand, an organocyclosiloxane of formula:

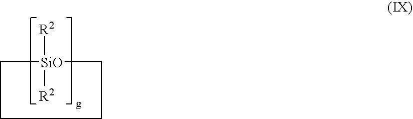

in which symbols R² are as defined above and g is a number ranging from 3 to 8, and optionally a dihydroxy POS of formula (VII). Such a process is suitable for preparing further organosilicon compounds comprising POSs of formula (III) in which the symbols T¹ and T² represent units HO_{1/2} and the symbol q is other than zero.

The organosilicon compounds which are preferably used in the context of the invention are those comprising polymers POS/1 of imide type. One advantageous procedure for preparing the organosilicon compounds comprising polymers POS/1 of imide type corresponds to a process (d) for preparing compounds comprising polymers POS/1 of imide type in the formula (III) of which the symbol q is equal to zero and consists in carrying out steps (d1) and (d2) below:

(d1) a reaction is carried out between:

an organosilane of formula (VI) in which the symbol X represents the function of formula (II/2), that is to say an organosilane of formula:

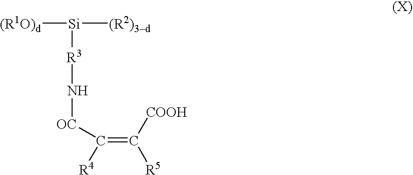

and a disilazane of formula:

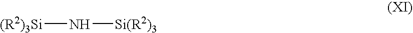

in which formulae the symbols R¹, R², R³, R⁴ and R⁵ are radicals corresponding to the definitions given in points (1) to (3) regarding formula (I) and d is a number chosen from 2 and 3,

this reaction being carried out in the presence of a catalyst, which may or may not be supported on a mineral material (such as, for example, a siliceous material), based on at least one Lewis acid, working at atmospheric pressure and at a temperature in the range from room temperature (23° C.) to 150° C. and preferably ranging from 60° C. to 120° C.;

(d2) stabilization of the reaction medium obtained is carried out by treating this medium with at least one halosilane of formula (R²)₃ Si-halo in which the halo residue is preferably chosen from a chlorine atom and a bromine atom, working in the presence of at least one non-nucleophilic organic base which is unreactive towards the imide function formed in situ during step (d1).

The disilazane is used in an amount at least equal to 0.5 mol per 1 mol of starting organosilane and preferably ranging from 1 to 5 mol per 1 mol of organosilane.

The preferred Lewis acid is ZnCl₂ and/or ZnBr₂ and/or Znl₂. It is used in an amount at least equal to 0.5 mol per 1 mol of organosilane and preferably ranging from 1 to 2 mol per 1 mol of organosilane.

The reaction is carried out in heterogeneous medium, preferably in the presence of a solvent or a mixture of solvents that are common with organosilicon reagents. The preferred solvents are of the polar aprotic type such as, for example, chlorobenzene, toluene, xylene, hexane, octane and decane. The solvents more preferably selected are toluene and xylene.

This process (d) may be carried out according to any procedure which is known per se. One procedure which is suitable is as follows: in a first stage, the reactor is fed with the Lewis acid and a solution of the organosilane in all or some of the solvent(s) is then gradually added; in a second stage, the reaction mixture is brought to the chosen temperature and the disilazane is then added, which may optionally be used in the form of a solution in some of the solvent(s); next, in a third stage, the reaction mixture obtained is treated with at least one halosilane in the presence of one or more organic base(s) in order to stabilize it; and finally, in a fourth step, the stabilized reaction medium is filtered to remove the Lewis acid and the salt formed in situ during the stabilization, and it is then devolatilized under reduced pressure to remove the solvent(s).

As regards the stabilization step (d2), the halosilane(s) is (are) used in an amount at least equal to 0.5 mol per 1 mol of starting organosilane and preferably ranging from 0.5 to 1.5 mol per 1 mol of organosilane. As regards the organic bases, the ones that are preferred are, in particular, tertiary aliphatic amines (such as, for example, N-methylmorpholine, triethylamine and triisopropylamine) and hindered cyclic amines (such as, for example, 2,2,6,6-tetraalkylpiperidines). The organic base(s) is (are) used in an amount at least equal to 0.5 mol per 1 mol of starting organosilane and preferably ranging from 0.5 to 1.5 mol per 1 mol of organosilane.

A second advantageous procedure, which may be used for preparing organosilicon compounds comprising polymers POS/1 of imide type, corresponds to a process (e) for preparing compounds comprising polymers POS/1 of imide type in the formula (III) of which the symbol q is other than zero, and consists in carrying out the single step (d1) defined as indicated above, but in which the disilazane of formula (XI) has been replaced with a cyclic polysilazane of formula:

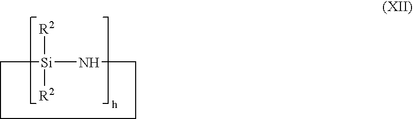

in which the symbols R² are as defined above and h is a number ranging from 3 to 8.

This process (e) may be carried out using the suitable procedure given above with regard to the implementation of process (d), and based on carrying out only the first stage, second stage and fourth stage mentioned above. It should be noted, however, that the polysilazane is used in an amount at least equal to 0.5/h mol per 1 mol of starting organosilane and preferably ranging from 1/h to 5/h mol per 1 mol of organosilane (h being the number of silazane units in the polysilazane of formula (XII)).

The implementation of processes (d) and (e) leads to the production of an organosilicon compound which may be in the form of a multifunctional POS in pure form or in the form of a mixture of a multifunctional POS with a variable weight amount (generally very much less than 50% in the mixture) of another (or of other) compound(s) which may consist, for example, of:

(i) a small amount of the unreacted starting organosilane of formula (X); and/or

(ii) a small amount of the organosilane of formula:

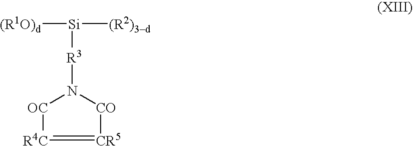

formed by direct cyclization of the corresponding amount of the starting organosilane of formula (X); and/or

(iii) a small amount of the cyclic monofunctional POS of formula:

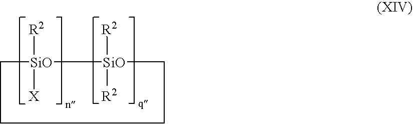

in which:

the symbols R² are as defined above in point (1) regarding formula (I),

the symbols X are as defined above in point (3) regarding formula (I),

the symbols n″ and q″ are integers or fractions which satisfy the following cumulative conditions:

n″ is in the range from 1 to 9,

q″ is in the range from 1 to 9,

the sum n″+q″ is in the range from 3 to 10,

the said cyclic monofunctional POS being derived from a modification of the silicone skeleton of the desired multifunctional POS.

### APPLICATION

The organosilicon compounds according to the invention, comprising the multifunctional POSs in accordance with formulae (I), (III) and (III′) given above, may be advantageously used as white filler-elastomer coupling agents in elastomer compositions of natural or synthetic rubber type based on isoprene elastomer(s), comprising a white filler, in particular a siliceous material, as reinforcing filler, these compositions being intended for manufacturing elastomeric articles.

The types of elastomeric articles for which the use of a coupling agent is most useful are those that are especially subject to the following constraints: large temperature variations and/or large variations in dynamic frequency stress; and/or a large static stress and/or a large dynamic bending fatigue. Examples of types of articles include: conveyor belts, power transmission belts, flexible tubes, expansion seals, seals on household electrical appliances, supports acting as engine vibration extractors either with metallic armouring or with a hydraulic fluid inside the elastomer, cables, cable sheaths, shoe soles and rollers for cable cars.

It is known by those skilled in the art that it is necessary to use a coupling agent, also known as a binder, whose function is to provide the connection between the surface of the particles of white filler and the elastomer, while at the same time making this white filler disperse more easily in the elastomer matrix.

The Applicant has discovered in its research that:

specific coupling agents consisting of a compound comprising a multifunctional POS in accordance with formulae (I), (III) and (III′), firstly bearing at least one OH radical and at least one alkoxy radical and secondly bearing at least one group containing an activated ethylenic double bond of maleimide type,

offer coupling performance qualities that are at least equivalent to those associated with the use of alkoxysilane polysulphides, in particular TESPT or bis(3-triethoxysilylpropyl) tetrasulphide which is generally considered nowadays as the product which gives, for silica filler vulcanizates, the best compromise in terms of safety from scorching, ease of use and reinforcing power, but which have the known drawback of being very expensive (see, for example, patents U.S. Pat. No. 5,562,310, U.S. Pat. No. 5,684,171 and U.S. Pat. No. 5,684,172),

when the said specific coupling agents are used in rubber compositions based on isoprene elastomer(s).

The elastomer compositions comprise:

at least one isoprene elastomer,

a white reinforcing filler, and

a suitable amount of a coupling agent consisting of the organosilicon compound comprising the multifunctional POS which has been defined above, firstly bearing at least one hydroxyl radical and/or at least one alkoxy radical, and secondly bearing at least one activated ethylenic double bond of maleimide type.

More specifically, these compositions comprise (the parts are given on a weight basis):

per 100 parts of isoprene elastomer(s),

from 10 to 150 parts of white filler, preferably from 30 to 100 parts and more preferably from 30 to 80 parts,

an amount of coupling agent or organosilicon compound which provides in the composition from 0.5 to 15 parts of multifunctional POS, preferably from 0.8 to 10 parts and more preferably from 1 to 8 parts.

Advantageously, the amount of coupling agent, chosen in the abovementioned general and preferred regions, is determined such that it represents from 1% to 20%, preferably from 2% to 15% and more preferably from 3% to 8% relative to the weight of the white reinforcing filler.

We will return in the text hereinbelow to the definitions, in turn, of the isoprene elastomers, and of the white reinforcing filler.

The term “isoprene elastomers”, which are used for the rubber compositions means, more specifically:

(1) synthetic polyisoprenes obtained by homopolymerization of isoprene or 2-methyl-1,3-butadiene;

(2) synthetic polyisoprenes obtained by copolymerization of isoprene with one or more ethylenically unsaturated monomers chosen from:

(2.1) conjugated diene monomers, other than isoprene, containing from 4 to 22 carbon atoms, such as, for example: 1,3-butadiene, 2,3-dimethyl-1,3-butadiene, 2-chloro-1,3-butadiene (or chloroprene), 1-phenyl-1,3-butadiene, 1,3-pentadiene, 2,4-hexadiene;

(2.2) vinylaromatic monomers containing from 8 to 20 carbon atoms, such as, for example: styrene, ortho-, meta- or para-methylstyrene, the “vinyltoluene” commercial mixture, para-tert-butylstyrene, methoxystyrenes, chlorostyrenes, vinylmesitylene, divinylbenzene, vinylnaphthalene;

(2.3) vinylic nitrile monomers containing from 3 to 12 carbon atoms, such as, for example, acrylonitrile and methacrylonitrile;

(2.4) acrylic ester monomers derived from acrylic acid or methacrylic acid with alkanols containing from 1 to 12 carbon atoms, such as, for example, methyl acrylate, ethyl acrylate, propyl acrylate, n-butyl acrylate, isobutyl acrylate, 2-ethylhexyl acrylate, methyl methacrylate, ethyl methacrylate, n-butyl methacrylate, isobutyl methacrylate;

(2.5) a mixture of several of the abovementioned monomers (2.1) to (2.4) together;

the polyisoprene copolymers containing between 99% and 20% by weight of isoprene units and between 1% and 80% by weight of diene, vinylaromatic, vinylic nitrile and/or acrylic ester units and consisting, for example, of poly(isoprene-butadiene), poly(isoprene-styrene) and poly(isoprene-butadiene-styrene);

(3) natural rubber;

(4) copolymers obtained by copolymerization of isobutene and isoprene (butyl rubber), as well as the halogenated versions, in particular chlorinated or brominated versions, of these copolymers;

(5) a mixture of several of the abovementioned elastomers (1) to (4) together;

(6) a mixture containing a major amount (ranging from 51% to 99.5% and preferably from 70% to 99% by weight) of abovementioned elastomer (1) or (3) and a minor amount (ranging from 49% to 0.5% and preferably from 30% to 1% by weight) of one or more diene elastomers other than isoprene elastomers.

The expression “diene elastomer other than an isoprene elastomer” means, in a manner which is known per se: the homopolymers obtained by polymerization of one of the conjugated diene monomers defined above in point (2.1), such as, for example, polybutadiene and polychloroprene; the copolymers obtained by copolymerization of at least two of the abovementioned conjugated dienes (2.1) together or by copolymerization of one or more of the abovementioned conjugated dienes (2.1) with one or more abovementioned unsaturated monomers (2.2), (2.3) and/or (2.4), such as, for example, poly(butadiene-styrene) and poly(butadiene-acrylonitrile).

Preferably, use is made of one or more isoprene elastomers chosen from: (1) synthetic polyisoprene homopolymers; (2) synthetic polyisoprene copolymers consisting of poly(isoprene-butadiene), poly(isoprene-styrene) and poly(isoprene-butadiene-styrene); (3) natural rubber; (4) butyl rubber; (5) a mixture of the abovementioned elastomers (1) to (4) together; (6) a mixture containing a major amount of abovementioned elastomer (1) or (3) and a minor amount of diene elastomer other than an isoprene elastomer, consisting of polybutadiene, polychloroprene, poly(butadiene-styrene) and poly(butadiene-acrylonitrile).

More preferably, use is made of one or more isoprene elastomers chosen from: (1) synthetic polyisoprene homopolymers; (3) natural rubber; (5) a mixture of the abovementioned elastomers (1) and (3); (6) a mixture containing a major amount of abovementioned elastomer (1) or (3) and a minor amount of diene elastomer other than an isoprene elastomer, consisting of polybutadiene and poly(butadiene-styrene).

In the present specification, the expression “white reinforcing filler” is intended to define a “white” (that is to say inorganic or mineral) filler, occasionally referred to as a “clear” filler, capable of reinforcing by itself, without any means other than that of a coupling agent, an elastomer composition of rubber type, which elastomer(s) may be natural or synthetic.

The white reinforcing filler may be in any physical state, that is to say that the said filler may be in the form of powder, micropearls, granules or beads.

Preferably, the white reinforcing filler consists of silica, alumina or a mixture of these two species.

More preferably, the white reinforcing filler consists of silica, taken alone or as a mixture with alumina.

Any precipitated or pyrogenic silica known to those skilled in the art, with a BET specific surface≦450 m²/g, is suitable as a silica which may be used in the invention. Precipitation silicas are preferred, these possibly being conventional or highly dispersible.

The expression “highly dispersible silica” means any silica which has a very strong ability to de-aggregate and to disperse in a polymer matrix, which may be observed by electron or optical microscopy, on thin slices. Non-limiting examples of highly dispersible silicas which may be mentioned include those with a CTAB specific surface of less than or equal to 450 m²/g and particularly those disclosed in patent U.S. Pat. No. 5,403,570 and patent applications WO-A-95/09127 and WO-A-95/09128, the content of which is incorporated herein. Treated precipitated silicas such as, for example, the aluminium-“doped” silicas disclosed in patent application EP-A-0 735 088, the content of which is also incorporated herein, are also suitable.

More preferably, precipitation silicas that are particular suitable are those with:

a CTAB specific surface ranging from 100 to 240 m²/g and preferably from 100 to 180 m²/g,

a BET specific surface ranging from 100 to 250 m²/g and preferably from 100 to 190 m²/g,

a DOP oil uptake of less than 300 ml/100 g and preferably ranging from 200 to 295 ml/100 g,

a BET specific surface/CTAB specific surface ratio ranging from 1.0 to 1.6.

Needless to say, the term “silica” also means blends of different silicas. The CTAB specific surface is determined according to NFT method 45007 of November 1987. The BET specific surface is determined according to the Brunauer-Emmet-Teller method described in “The Journal of the American Chemical Society, vol. 80, page 309 (1938)” corresponding to NFT standard 45007 of November 1987. The DOP oil uptake is determined according to NFT standard 30-022 (March 1953) using dioctyl phthalate.

The reinforcing alumina advantageously used is a highly dispersible alumina with:

a BET specific surface ranging from 30 to 400 m²/g and preferably from 80 to 250 m²/g,

an average particle size of not more than 500 nm and preferably not more than 200 nm, and

a high content of reactive Al—OH surface functions, as disclosed in document EP-A-0 810 258.

Non-limiting examples of such reinforcing aluminas which will be mentioned in particular include the aluminas A125, CR125 and D65CR from the company Baïkowski.

Needless to say, the compositions of rubber type also contain all or some of the other additional constituents and additives usually used in the field of elastomer compositions and rubber compositions.

Thus, all or some of the other constituents and additives below may be used:

as regards the vulcanization system, mention will be made, for example, of:

vulcanizing agents chosen from sulphur and sulphur-donating compounds such as, for example, thiuram derivatives;

vulcanization accelerators such as, for example, guanidine derivatives, thiazole derivatives or sulphenamide derivatives;

vulcanization activators such as, for example, zinc oxide, stearic acid and zinc stearate;

as regards other additive(s), mention will be made, for example, of:

a conventional reinforcing filler such as carbon black (in this case, the white reinforcing filler used constitutes more than 50% of the total weight of white reinforcing filler+carbon black);

a conventional white filler which provides little or no reinforcement, such as, for example, clays, bentonites, talc, chalk, kaolin, titanium dioxide or a mixture of these species;

antioxidants;

anti-ozonizers such as, for example, N-phenyl-N′-(1,3-dimethylbutyl)-p-phenylenediamine;

plasticizers and processing adjuvants.

The vulcanization (or curing) of the rubber compositions is carried out in a known manner at a temperature generally ranging from 130° C. to 200° C., for a sufficient time which can range, for example, between 5 and 90 minutes depending in particular on the curing temperature, the vulcanization system used and the vulcanization kinetics of the composition under consideration.

The examples which follow illustrate the present invention.

### EXAMPLE 1

This example illustrates the preparation of an organosilicon compound according to the invention, comprising a polymer POS/1 of imide type.

This compound is prepared by carrying out process (d) outlined above in the present specification, with, as starting organosilane of formula (X), N-[γ-propyl(methyidiethoxy)silane]maleamic acid.

1. Preparation of the Starting Maleamic Acid Silane

The process is performed in a 2-liter glass reactor equipped with a stirring system and an addition funnel. The γ-aminopropylsilane of formula (C₂H₅O)₂CH₃Si(CH₂)₃NH₂ (244.82 g, i.e. 1.28 mol) is gradually added at a temperature of 20° C. (reaction temperature maintained at this value by means of an ice-water bath placed under the reactor) to a solution of maleic anhydride (128.2 g, i.e. 1.307 mol) in toluene as solvent (442.5 g), over a period of 105 minutes. The reaction medium is then left at 23° C. for 15 hours. At the end of this time, the reaction medium is filtered through a sinter funnel of porosity 3 and a solution of the desired maleamic acid silane in toluene is thus recovered, which solution is used in the form in which it is obtained, to carry out the following process (d). This solution contains 0.157 mol of maleamic acid silane per 100 g of solution.

### 2. Preparation of the Organosilicon Compound Comprising a Polymer POS/1 of Imide Type by Carrying Out Process (d):

1st stage: ZnCl₂ (43.78 g, i.e. 0.3214 mol) is introduced into a 0.5-liter glass reactor equipped with a stirring system and an addition funnel and the solid is then heated at 80° C. for 1 hour 30 minutes under a reduced pressure of 3×10² Pa; the reactor is returned to atmospheric pressure, working under an argon atmosphere, and 91.45 g of the solution of maleamic acid silane (41.5 g, i.e. 0.143 mol) in toluene, obtained previously in point 1, are then added gradually;

2nd stage: the reaction mixture is brought to a temperature of 54° C. and hexamethyldisilazane (65.12 g, i.e. 0.403 mol) is then added gradually over a period of one hour; at the end of the addition, the temperature of the reaction medium is 82° C., and is maintained at this value for a further 1 hour 30 minutes;

3rd stage: N-methylmorpholine (20.14 g, i.e. 0.199 mol) is introduced into the reaction medium, followed by trimethylchlorosilane (21.49 g, i.e. 0.198 mol), working at a temperature of about −20° C.; the resulting reaction medium is left stirring for 15 hours, while allowing the temperature to rise slowly to room temperature (23° C.);

4th stage: the reaction medium obtained is filtered through a sinter funnel of porosity 3 containing 2 cm of silica, and the filtrate obtained is then devolatilized at 30° C. by establishing a reduced pressure of 10×10² Pa, to give a brown oil comprising the desired oligomer POS/1 of imide type. The said brown oil was subjected to proton NMR and silicon (²⁹Si) NMR analyses. The results of these analyses reveal that the reaction product or organosilicon compound obtained after process (d) contains:

62% by weight of polymer POS/1 of imide type in the form of an oligomer of average formula:

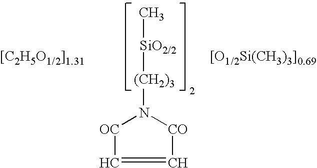

and 38% by weight of the organosilane of formula:

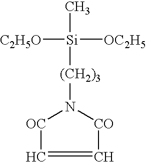

### EXAMPLE 2

This example illustrates the preparation of an organosilicon compound according to the invention, comprising another polymer POS/1 of imide type.

This other compound is prepared by carrying out process (e) which was outlined above in the present specification, with N-[γ-propyl(methyldiethoxy)silane]maleamic acid as starting organosilane of formula (X).

### 1. Preparation of the Starting Maleamic Acid Silane

The process is performed in a 2-liter glass reactor equipped with a stirring system and an addition funnel. The γ-aminopropylsilane of formula (C₂H₅O)₂CH₃Si(CH₂)₃NH₂ (563 g, i.e. 2.944 mol) is gradually added at a temperature of 20-22° C. (reaction temperature maintained at this value by means of an ice-water bath placed under the reactor) to a solution of maleic anhydride (300.1 g, i.e. 3.062 mol) in toluene as solvent (1008 g), over a period of 2 hours. The reaction medium is then left at 23° C. for 15 hours. At the end of this time, the reaction medium is filtered through a sinter funnel of porosity 3 and a solution of the desired maleamic acid silane in toluene is thus recovered, which solution is used in the form in which it is obtained, to carry out the following process (e). This solution contains 0.157 mol of maleamic acid silane per 100 g of solution.

### 2. Preparation of the Organosilicon Compound Comprising Another Polymer POS/1 of Imide type by Carrying out Process (e)

1st stage: ZnCl₂ (168.2 g, i.e. 1.2342 mol) is introduced into a 3-liter glass reactor equipped with a stirring system and an addition funnel, and the solid is then heated at 80° C. for 1 hour 30 minutes under a reduced pressure of 4×10² Pa; the reactor is then returned to atmospheric pressure, working under an argon atmosphere, and 365 cm³ of toluene are then added, followed by gradual addition of 704.8 g of the solution of maleamic acid silane (320 g, i.e. 1.107 mol) in toluene which was obtained previously in point 1;

2nd stage: the addition funnel is loaded with cyclic hexamethyltrisilazane (88.7 g, i.e. 0.404 mol) and 208 cm³ of toluene; the temperature of the reaction medium is 72° C. The cyclic hexamethyltrisilazane is then added gradually over a period of 2 hours 25 minutes; at the end of the addition, the orange-coloured organic solution obtained is heated to a temperature of 75° C. and is maintained at this temperature for 15 hours;

4th stage: the reaction medium is filtered through a “cardboard filter” and the toluene is then removed after devolatilization under reduced pressure.

A yellow oil is thus obtained, which was subjected to proton NMR and silicon (²⁹Si) NMR analyses. The results of these analyses reveal that the reaction product or organosilicon compound obtained after process (e) contains:

73.7% by weight of polymer POS/1 of imide type in the form of an oligomer of average formula:

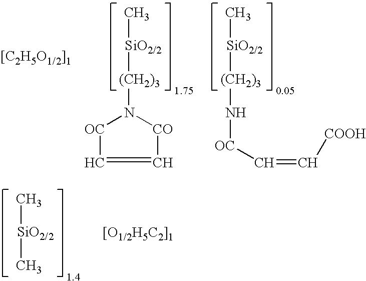

23.1% by weight of the organosilane of formula:

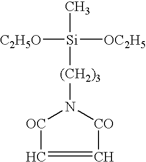

0.7% by weight of the organosilane of formula:

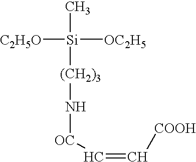

and 2.5% by weight of the cyclic monofunctional POS of average formula:

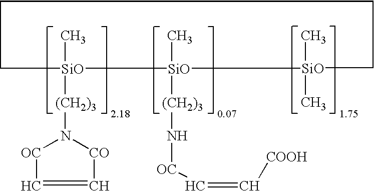

### EXAMPLES 3 AND 4

The aim of these examples is to show the performance qualities in terms of coupling (for white filler-isoprene elastomer coupling) of an organosilicon compound comprising a multifunctional POS which was defined above, firstly bearing at least one hydroxyl radical and/or at least one alkoxy radical, and secondly bearing at least one activated ethylenic double bond of maleimide type. These performance qualities are compared with those of a conventional coupling agent based on a TESPT silane.

Four isoprene elastomer compositions representative of shoe sole formulations are compared. These 4 compositions are identical except for the following differences:

composition No. 1 (control 1): absence of coupling agent;

composition No. 2 (control 2): coupling agent based on TESPT silane (4 pce);

composition No. 3 (Example 3): coupling agent or organosilicon compound providing in the composition 1.86 pce of polymer POS/1 of imide type, prepared in Example 1;

composition No. 4 (Example 4): coupling agent or organosilicon compound providing in the composition 2.65 pce of polymer POS/1 of imide type, prepared in Example 2.

### 1) Constitution of the Isoprene Elastomer Compositions

The compositions below, the constitution of which, expressed in parts by weight, is given in Table I given below, are prepared in a Brabender internal mixer:

TABLE 1

| | Control | Control | Ex. | Ex. |
|---|---|---|---|---|
| Composition | 1 | 2 | 3 | 4 |
| NR rubber (1) | 85 | 85 | 85 | 85 |
| BR 1220 rubber (2) | 15 | 15 | 15 | 15 |
| Silica (3) | 50 | 50 | 50 | 50 |
| Zinc oxide (4) | 5 | 5 | 5 | 5 |
| Stearic acid (5) | 2 | 2 | 2 | 2 |
| TESPT silane (6) | ,— | 4 | — | — |
| Organosilicon compound comprising | — | — | 3 | — |
| the polymer POS/1 of imide type pre- | | | | |
| pared in Example 1 | | | | |
| Organosilicon compound comprising | — | — | — | 3.6 |
| the polymer POS/1 of imide type | | | | |
| prepared in Example 2 | | | | |
| TBBS (7) | | | | |
| DPG (8) | | | | |
| Sulphur (9) | 2 | 2 | 2 | 1 |
| | 1.4 | 1.4 | 1.4 | 1.4 |
| | 1.7 | 1. | 7 1.7 | 1.7 |

(1) Natural rubber, of Malaysian origin, sold by the company Safic-Alcan under the reference SMR 5L;
(2) Polybutadiene rubber with a high content of cis-1,4 addition products, sold by the company Shell;
(3) Zéosil 1165 MP silica, sold by the company Rhodia-Silices;
(4) and (5) Vulcanization activators;
(6) Bis(3-triethoxysilylpropyl) tetrasulphide sold by the company Degussa under the name Si-69;
(7) N-tert-Butyl-2-benzothiazylsulphenamide (vulcanization accelerator);
(8) Diphenylguanidine (vulcanization accelerator);
(9) Vulcanizing agent.

### 2) Preparation of the Compositions

The various constituents are introduced, in order, at the times and temperatures given below, into a Brabender internal mixer:

| Time | Temperature | Constituents |
|---|---|---|
| 0 minute | 80° C. | NR rubber |
| 1 minute | 90° C. | BR rubber |
| 2 minutes | 100° C. | ⅔ silica + coupling agent |
| 4 minutes | 120° C. | ⅓ silica + stearic acid + zinc oxide |
| | | Discharge |
| 5 minutes | 140 to 150° C. | |

The discharge or sedimentation of the contents of the mixer takes place after 5 minutes. The temperature reached is approximately 145° C.

The mixture obtained is then introduced into a roll mill, maintained at 30° C., and the TBBS, the DPG and the sulphur are introduced. After homogenization, the final mixture is calendered in the form of sheets 2.5 to 3 mm thick.

### 3) Rheological Properties of the Compositions

The measurements are taken on the compositions in raw form. The results regarding the rheology test which is carried out at 160° C. for 30 minutes using a Monsanto 100 S rheometer are given in Table II below.

According to this test, the test composition is placed in the test chamber adjusted to a temperature of 160° C., and the resistant torque, opposed by the composition, to an oscillation of low amplitude of a biconical rotor included in the test chamber is measured, the composition completely filling the chamber under consideration. From the curve of variation of the torque as a function of time, the following are determined: the minimum torque which reflects the viscosity of the composition at the temperature under consideration; the maximum torque and the delta-torque which reflect the degree of crosslinking entailed by the action of the vulcanization system; the time T-90 required to obtain a vulcanization state corresponding to 90% of the complete vulcanization (this time is taken as the vulcanization optimum); and the scorch time TS-2 corresponding to the time required for a 2-point increase above the minimum torque at the temperature under consideration (1 60° C.) and which reflects the time for which it is possible to use the raw mixtures at this temperature without any initiation of vulcanization taking place.

The results obtained are given in Table II.

TABLE II

| | Control | Control | Example | Example |
|---|---|---|---|---|
| Monsanto rheology | 1 | 2 | 3 | 4 |
| Minimum torque | 27.1 | 15.3 | 18.2 | 15.7 |
| Maximum torque | 81.5 | 108.5 | 92.8 | 97.8 |
| Delta-torque | 54.4 | 93.2 | 74.6 | 82.1 |
| TS-2 (minutes) | 4 | 3.6 | 3.1 | 2.5 |
| TS-90 (minutes) | 7.4 | 7.33 | 6.4 | 5.69 |

### 4) Mechanical Properties of the Vulcanizates

The measurements are taken on compositions uniformly vulcanized for 20 minutes at 160° C.

The properties measured and the results obtained are collated in Table III below:

TABLE III

| | Con- | Con- | Ex. | Ex. |
|---|---|---|---|---|
| Mechanical properties | trol 1 | trol 2 | 3 | 4 |
| 10% Modulus (1) | 0.65 | 0.89 | 0.75 | 0.81 |
| 100% Modulus (1) | 1.31 | 3.54 | 2.56 | 2.9 |
| 300% Modulus (1) | 3.7 | 15.2 | 12.3 | 14.1 |
| Elongation at break (1) | 810 | 370 | 480 | 400 |
| Breaking strength (1) | 23.8 | 19 | 24 | 21 |
| Reinforcement indices: | 2.8 | 4.3 | 4.8 | 4.9 |
| 300% M/100% M | | | | |
| Shore A hardness (2) | 65 | 74 | 70 | 70 |
| Abrasion resistance (3) | 227 | 113 | 89 | 90 |

(1) The tensile tests are performed in accordance with the indications of NF T standard 46-002 with test pieces of H2 type. The 10%, 100% and 300% moduli and the breaking strength are expressed in MPa; the elongation at break is expressed in %.
(2) The measurement is taken according to the indications of ASTM standard D 3240. The value given is measured at 15 seconds.
(3) The measurement is taken according to the indications of NF T standard 46-012, using method 2 with a rotating sample holder. The value measured is the loss of substance (in mm³) on abrasion; the lower the value, the better the abrasion resistance.

It is found that, after curing, the compositions of Examples 3 and 4 show modulus values under high deformation (300% M) and reinforcement indices which are higher than those of the control mixture without coupling agent and which may be higher than those obtained with the TESPT silane (control 2).

It is also noted that all the mixtures mentioned show an abrasion resistance which is very substantially greater than that of control 1.

The improvement in these indicators is known to those skilled in the art as demonstrating a significant improvement in the white filler-elastomer coupling due to an incontestable coupling effect of the coupling agents introduced into the compositions of Examples 3 and 4.

It is pointed out most particularly that the coupling agent used in Example 4 (compound comprising the polymer POS/1 of imide type prepared in Example 2) leads to a particularly advantageous compromise of properties since it makes it possible simultaneously to obtain:

viscosities similar to those achieved with TESPT (control 2),

a 300% modulus which is quite close to that imparted by TESPT,

a reinforcement index which is substantially higher than that obtained with TESPT,

an excellent level of abrasion resistance, which is substantially better than that imparted by TESPT.

1. Novel organosilicon compounds which comprise multifunctional POSs, characterized in that the said multifunctional POSs contain identical or different units of formula:
$${( {R}^{2} )}_{a} {Y}_{b} {X}_{c} {SiO}_{\frac{4 - ( a + b + c )}{2}} \qquad ( I )$$
in which:
(1) the symbols R², which may be identical or different, each represent a monovalent hydrocarbon-based group chosen from a linear or branched alkyl radical containing from 1 to 6 carbon atoms, a cycloalkyl radical containing from 5 to 8 carbon atoms and a phenyl radical;
(2) the symbols Y, which may be identical or different, each represent a hydroxyl or alkoxy function R¹O in which R¹ represents a linear or branched alkyl radical containing from 1 to 15 carbon atoms;
(3) the symbols X, which may be identical or different, each represent a function bearing an activated ethylenic double bond, chosen from the radicals having the formulae (II/1), (II/2) and (II/3) below, and mixtures thereof: 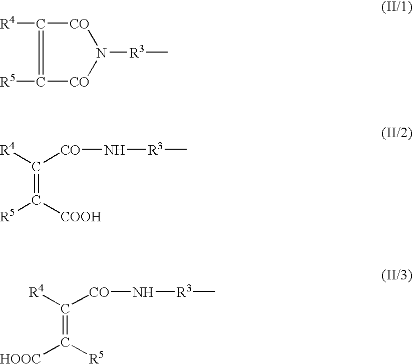
with the conditions according to which:
at least one of the functions X corresponds to formula (II/1),
when, where appropriate, there is a mixture of function(s) X of formula (II/1) with functions X of formulae (II/2) and/or (II/3), the mole fraction of functions X of formulae (II/2) and/or (II/3) in all of the functions X is on average less than or equal to 12 mol %,
formulae in which:
R³ is a linear or branched divalent alkylene radical containing from 1 to 15 carbon atoms, the free valency of which is borne by a carbon atom and is linked to a silicon atom, the said radical R³ possibly being interrupted in the alkylene chain with at least one hetero atom (such as oxygen and nitrogen) or at least one divalent group comprising at least one hetero atom (such as oxygen and nitrogen), and in particular with at least one divalent residue of general formula ^{V1}radical^{V2}chosen from: —O—, —CO—, —CO—O—, —COO-cyclohexylene (optionally substituted with an OH radical)-, —O-alkylene (linear or branched C₂-C₆, optionally substituted with an OH or COOH radical)-, —O—CO-alkylene (linear or branched C₂-C₆, optionally substituted with an OH or COOH radical)-, —CO—NH—, O—CO—NH— and —NH-alkylene (linear or branched C₂-C₆)—CO—NH—; R³ also represents a divalent aromatic radical of general formula ^{V1}radical^{V2}chosen from: -(ortho, meta or para)phenylene(linear or branched C₂-C₆)alkylene-, -(ortho, meta or para)phenylene-O-(linear or branched C₂-C₆)alkylene-, -(linear or branched C₂-C₆)alkylene-(ortho, meta or para)phenylene(linear or branched C₁-C₆)alkylene-, and -(linear or branched C₂-C₆)alkylene(ortho, meta or para)phenylene-O-(linear or branched C₁-C₆)alkylene-; preferably, the symbol R³ represents an alkylene radical which corresponds to the following formulae: —(CH₂)₂—, —(CH₂)₃—, —(CH₂)₄—, —CH₂—CH(CH₃)—, —(CH₂)₂—CH(CH₃)—CH₂—, —(CH₂)₃—O—(CH₂)₃—, —(CH₂)₃—O—CH₂—CH(CH₃)—CH₂—, —(CH₂)₃—O—CH₂CH(OH)—CH₂—; more preferably, R³ is a —(CH₂)₂— or —(CH₂)₃-radical; with the specific detail that, in the preceding definitions of R³, when the divalent residues and radicals mentioned are not symmetrical, they may be positioned with the valency v1 to the left and the valency v2 to the right, or vice versa with the valency v2 to the left and the valency v1 to the right;
the symbols R⁴ and R⁵, which may be identical or different, each represent a hydrogen atom, a halogen atom, a cyano radical, a linear or branched alkyl radical containing from 1 to 6 carbon atoms;
(4) the symbols a, b and c each represent integers or fractions chosen from:
a: 0, 1, 2or 3;
b: 0, 1, 2or 3;
c: 0 or 1;
the sum a+b+c being other than zero and ≦3;
(5) the content of units R⁶SiO_{3/2} (units “T”) in which R⁶ is chosen from the radicals corresponding to the definitions of R², Y and X, this content being expressed as the number, per molecule, of these units per 100 silicon atoms, is less than or equal to 30%;
(6) the content of functions Y, expressed as the number, per molecule, of functions Y per 100 silicon atoms, is at least 0.8%;
(7) the content of functions X, expressed as the number, per molecule, of functions X per 100 silicon atoms, is at least 0.4%.

2. Compounds according to claim 1, characterized in that, in formula I:
(1) the symbols R² are chosen from methyl, ethyl, n-propyl, isopropyl, n-butyl, n-pentyl, cyclohexyl and phenyl radicals;
(2) the symbols Y are chosen from a hydroxyl radical and a linear or branched alkoxy radical containing from 1 to 6 carbon atoms;
(3) the functions represented by the symbol X are chosen from the functions of formulae (II/1), (II/2) and (II/3), and mixtures thereof, in which:
when, where appropriate, there is a mixture of function(s) X of formula (II/1) with functions X of formulae (II/2) and/or (II/3), the mole fraction of functions X of formula (II/2) and/or (II/3) in all of the functions X is on average less than or equal to 5 mol %;
the symbol R³ represents an alkylene radical which corresponds to the following formulae: —(CH₂)₂—, —(CH₂)₃—, —(CH₂)₄—, —CH₂—CH(CH₃)—, —(CH₂)₂—CH(CH₃)—CH₂—, —(CH₂)₃—O—(CH₂)₃—, —(CH₂)₃—O—CH₂—CH(CH₃)—CH₂— and —(CH₂)₃—O—CH₂CH(OH)—CH₂—;
the symbols R⁴ and R⁵ are chosen from a hydrogen atom, a chlorine atom and methyl, ethyl, n-propyl, and n-butyl radicals;
(5) the content of units “T” is less than or equal to 20%;
(6) the content of functions Y is in the range from 1% to 100%;
(7) The content of functions X is in the range from 0.8% to 100%.

3. Compounds according to claim 1 or 2, characterized in that they comprise multifunctional POSs chosen from:
POSs which are essentially linear and which have the average formula below: 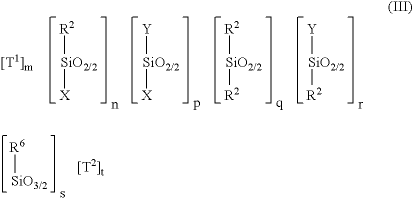
in which:
(1′) the symbols T¹ are chosen from the units HO_{1/2} and R¹O_{1/2}, in which the radical R¹ is as defined above;
(2′) the symbols T², which may be identical to or different from the symbols T¹, are chosen from the units HO_{1/2} and R¹O_{1/2} and the unit (R²)₃SiO_{1/2}, in which the radicals R¹ and R² are as defined above in points (2) and (1) regarding formula (I);
(3′) the symbols R², X and Y are as defined above in points (1), (3) and (2) regarding formula (I);
(4′) the symbols R⁶ are chosen from the radicals corresponding to the definitions of R², X and Y;
(5′) the symbols m, n, p, q, r, s and t each represent integers or fractions which satisfy the following cumulative conditions:
m and t are each numbers that are always other than zero, the sum of which is equal to 2+s,
n is in the range from 0 to 100,
p is in the range from 0 to 100,
q is in the range from 0 to 100,
r is in the range from 0 to 100,
s is in the range from 0 to 75,
when n=0, p is always a number other than 0 and when p=0, n is always a number other than zero,
the sum n+p+q+r+s+t giving the total number of silicon atoms is in the range from 2 to 250,
the ratio 100 s/(n+p+q+r+s+t) giving the content of units “T” is ≦30,
the ratio 100 (m+p+r+s [when R⁶=Y]+t)/(n+p+q+r+s+t) giving the content of functions Y is ≧1,
the ratio 100 (n+p+s [when R⁶=X])/(n+p+q+r+s+t) giving the content of functions X is ≧1;
POSs which are cyclic and which have the average formula below: 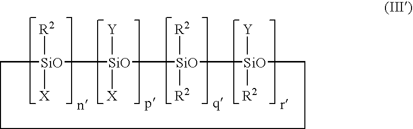
in which:
(3′) the symbols R², X and Y are as defined above in points (1), (3) and (2) regarding formula (I);
(5′) the symbols n′, p′, q′ and r′ each represent integers or fractions which satisfy the following cumulative conditions:
n′ is in the range from 0 to 9,
p′ is in the range from 0 to 9,
when n′=0, p′ is at least equal to 1,
when p′=0, n′ is at least equal to 1 and r′ is also at least equal to 1,
q′ is in the range from 0 to 9,
r′ is in the range from 0 to 2,
the sum n′+p′+q′+r′ is in the range from 3 to 10,
the ratio 100 (p′+r′)/(n′+p′+q′+r′) giving the content of functions Y ranges from 4 to 100,
the ratio 100 (n′+p′)/(n′+p′+q′+r′) giving the content of functions X ranges from 10 to 100.
and mixtures of the POSs of formulae (III) and (III′).

4. Compounds according to claim 3, characterized in that they comprise multifunctional POSs chosen from the essentially linear oligomers and polymers POS/1 which correspond to formula (III) in which:
(1″) the symbols T¹ are defined as given above in point (1′);
(2″) the symbols T² are defined as given above in point (2′);
(3″) the symbols R², X and Y are defined as given above in point (3′);
(4″) the symbols R⁶ are defined as given above in point (4′);
(5″) the symbols m, n, p, q, r, s and t satisfy the following cumulative conditions:
m+t=2+s,
n is in the range from 0 to 50,
p is in the range from 0 to 20,
when n=0, p is at least equal to 1 and when p=0, n is at least equal to 1,
q is in the range from 0 to 48,
r is in the range from 0 to 10,
s is in the range from 0 to 1,
the sum n+p+q+r+s+t giving the total number of silicon atoms is in the range from 2 to 50,
the ratio 100 s/(n+p+q+r+s+t) giving the content of units “T” is ≦10,
the ratio 100 (m+p+r+s [when R⁶=Y]+t)/(n+p+q+r+s+t) giving the content functions Y ranges from 4 to 100,
the ratio 100 (n+p+s [when R⁶=X])/(n+p+q+r+s+t) giving the content functions X ranges from 10 to 100.

5. Process for preparing the organosilicon compounds according to any one of claims 1 to 4, characterized in that it involves, in particular:
a hydrolysis and condensation reaction of a dihalosilane or of a dialkoxysilane bearing a function X, optionally in the presence of a dihalosilane or of a dialkoxysilane,
a condensation reaction between an organosilane bearing a function X and at least two functions Y, and an α,ω-dihydroxylated linear POS,
a redistribution and equilibration reaction between an organosilane bearing a function X and at least two functions Y and/or halo, and an organocyclosiloxane optionally bearing one or more functions Y in the chain,
a coupling reaction between an organosilane bearing a function X of formula (II/2) and at least two functions Y, and a polysilazane,
a coupling reaction between a linear or cyclic precursor POS bearing at least one function Y and functionalized with at least one unit attached to a silicon atom, in particular of -(linear or branched C₂-C₆)alkylene-OH, -(linear or branched C₂-C₆)alkylene-NR⁶H or -(linear or branched C₂-C₆)alkylene-COOH type, and a reactive compound capable of reacting with the abovementioned unit(s) to generate the desired function X.

6. Preparation process according to claim 5, for preparing the organosilicon compounds according to claim 4 and comprising the polymers POS/1 in the formula (III) of which the symbol q is equal to zero, characterized in that it consists in carrying out steps (d1) and (d2) below:
(d1) a reaction is carried out between:
an organosilane of formula (VI) in which the symbol X represents the function of formula (II/2), that is to say an organosilane of formula: 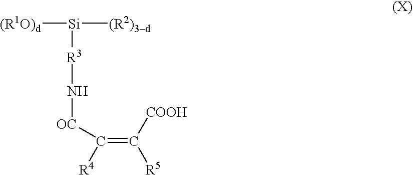
and a disilazane of formula: 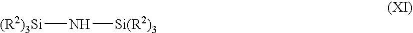
in which formulae the symbols R¹, R², R³, R⁴ and R⁵ are radicals corresponding to the definitions given in points (1) to (3) regarding formula (I) according to claim 1 and d is a number chosen from 2 and 3,
this reaction being carried out in the presence of a catalyst, which may or may not be supported on a mineral material, based on at least one Lewis acid, working at atmospheric pressure and at a temperature in the range from room temperature (23° C.) to 150° C.;
(d2) stabilization of the reaction medium obtained is carried out by treating this medium with at least one halosilane of formula (R²)₃ Si-halo, working in the presence of at least one non-nucleophilic organic base which is unreactive towards the imide function formed in situ during step (d1).

7. Preparation process according to claim 5, for preparing the organosilicon compounds according to claim 4 and comprising the polymers POS/1 in the formula (III) of which the symbol q is other than zero, characterized in that it consists in reacting:
an organosilane of formula (VI) in which the symbol X represents the function of formula (II/2), that is to say an organosilane of formula: 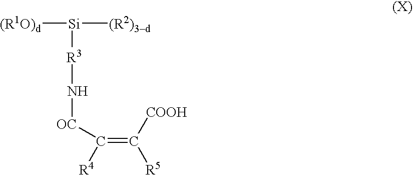
with a polysilazane of formula: 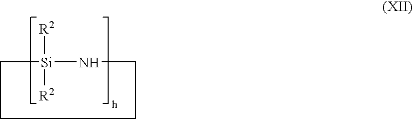
in which formulae the symbols R¹, R², R³, R⁴ and R⁵ are radicals corresponding to the definitions given in points (1) to (3) regarding formula (I) according to claim 1, and h is a number ranging from 3 to 8,
this reaction being carried out in the presence of a catalyst, which may or may not be supported on a mineral material, based on at least one Lewis acid, working at atmospheric pressure and at a temperature in the range from room temperature (23° C.) to 150° C.

8. Preparation process according to claim 6, characterized in that the operating conditions below are used:
the disilazane (XI) is used in an amount at least equal to 0.5 mol per 1 mol of starting organosilane (X);
the Lewis acid is used in an amount at least equal to 0.5 mol per 1 mol of starting organosilane (X);
the halosilane(s) of the stabilization step is (are) used in an amount at least equal to 0.5 mol per 1 mol of starting organosilane (X);
the organic base(s) of the stabilization step is (are) used in an amount at least equal to 0.5 mol per 1 mol of starting organosilane (X).

9. Preparation process according to claim 7, characterized in that the operating conditions below are used:
the polysilazane (XII) is used in an amount at least equal to 0.5/h mol per 1 mol of starting organosilane (X), h being the number of silazane units in the polysilazane of formula (XII);
the Lewis acid is used in an amount at least equal to 0.5 mol per 1 mol of starting organosilane (X).
